(19) United States
(12) Patent Application Publication
(10) Pub. No.: US 2004/0071648 A1
(43) Pub. Date: Apr. 15, 2004

(54) Cosmetic composition having a pasty to pulverulent texture and the cosmetic use thereof

(76) Inventors: Marie-Laure Delacour, Paris (FR); Patrice Styczen, Gif-sur-Yvette (FR); Xavier Ray, Villeconin (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/614,016

(22) Filed: Jul. 8, 2003

(30) Foreign Application Priority Data
Jan. 8, 2002 (WO) PCT/FR02/00044
Jan. 8, 2001 (FR) 01 00180
Oct. 3, 2001 (FR) 01 12733

(51) Int. Cl. A61K 7/06; A61K 7/11

(52) U.S. Cl. 424/70.12

(57) ABSTRACT

A novel cosmetic composition, and methods of use thereof, wherein the composition has a pasty to pulverulent texture, comprising at least one binder A and at least one particulate phase B, the binder A comprising at least water and particles of at least one partially or completely crosslinked elastomeric solid organopolysiloxane C, wherein the weight ratio of the at least one organopolysiloxane C to the at least one particulate phase B ranges from 0.4:1 to 1.8:1.

This application is a continuation-in-part of international application no. PCT/FR02/00044, filed Jan. 8, 2002, and now published with the international publication no. WO 02/053126.

Disclosed herein is a cosmetic, such as a make-up or care, composition in pasty and/or pulverulent form, for example in loose, compact or pressed form.

Make-up powders may generally comprise on the one hand, a particulate phase containing pigments and fillers and, on the other hand, a fatty phase as binder comprising fatty substances, which is intended to confer on the finished product a certain cohesion, to give softness and an emollient property to the make-up product and/or to promote its adhesion to the skin.

The formulation of binding agents in powders, such as in compact powders, may cause many difficulties because the final composition should be sufficiently homogeneous and compact in order to have a good capacity to be collected and to avoid, moreover, fragmentation caused, for example, by impact.

Make-up powders are therefore products which may generally comprise a very high amount of dry pulverulent compounds and oils. These products can give sensations of tightness or a drying effect when they are applied to the skin, and may provide no sensation of freshness.

Moreover, there have been described in patent applications EP-1,023,893 and EP 1,025,837 make-up or care compositions comprising particles of a hydrophilic organopolysiloxane. In the composition fields described, these compositions can be liquids which flow and which cannot therefore be shaped.

The inventors have found, unexpectedly, that the use of a binder comprising water and particles of an at least partially crosslinked elastomeric solid organopolysiloxane makes it possible to obtain a composition with a novel texture, from pasty to pulverulent, which exhibits excellent cosmetic properties, for example, in terms of freshness. Furthermore, such compositions can be advantageously shaped by pressing in a customary cosmetic container.

Disclosed herein, therefore, is a novel cosmetic, such as make-up or care, composition comprising at least one binder A and at least one particulate phase B, the binder A comprising at least water and particles of at least one at least partially crosslinked elastomeric solid organopolysiloxane C, wherein the weight ratio C/B of the at least one organopolysiloxane C to the at least one particulate phase B ranges from 0.4:1 to 1.8:1, such as from 0.4:1 to 1.5:1, for example, from 0.4:1 to 1.3:1, and further, for example, from 0.6:1 to 1.3:1. The weight ratio A/B of the at least one binder A to the at least one particulate phase B ranges from 0.6:1 to 3:1, for example, from 0.6:1 to 2.5:1, and further, for example, from 1:1 to 2.5:1.

In one embodiment, the composition comprises from 25 to 60% by weight of the at least one particulate phase B, relative to the total weight of the composition. For example, the composition may comprise from 35 to 60% by weight of the at least one particulate phase B, or further, for example, from 35 to 55% by weight, relative to the total weight of the composition. The particulate phase B does not include the particles of the elastomeric organopolysiloxane C, which are contained instead in the binder A.

The composition also comprises, in one embodiment, from 25 to 45% by weight of the at least one organopolysiloxane C, relative to the total weight of the composition. The organopolysiloxane plays the role of supple interparticulate “cushion.”

When it is a care composition, the composition disclosed herein is advantageously a care composition which modifies the appearance of the surface of the skin, for instance, by optical effects. For example, it may be a mattifying composition and/or a composition which masks skin imperfections such as redness, fine lines and/or pores. If this is the case, the composition is then referred to as a care composition with a mattifying effect.

The composition disclosed herein has a novel pulverulent (powdery) to pasty texture. The hardness and elasticity of the composition are measured at 20° C. using a texturometer sold under the name TA-XT2i by the Rheo Company, equipped with a stainless steel ball with a diameter of 12.7 mm by measuring the evolution of the force (force of compression or force of stretching) (F) as a function of time, during the following operation: the ball is moved at the speed of 0.1 mm/s, then penetrates into the product to a penetration depth of 0.3 mm. When the ball has penetrated to the depth of 0.3 mm, the ball is withdrawn at a speed of 0.1 mm/s. As the ball is withdrawn, the force (force of compression) strongly decreases until becoming zero at the end of a time t. The total time it takes the ball to penetrate 0.3 mm and then be removed from the product again (one cycle) is 6 seconds.

The hardness corresponds to the maximum force of compression measured during the operation and is expressed in Newtons. The hardness of the composition of the invention may range from 0.03 N to 0.7 N, such as from 0.05 N to 0.6 N, for example, from 0.07 to 0.4 N, and further, for example, from 0.1 to 0.35 N.

The elasticity El is expressed as a percentage determined by the formula:

El(%)=100×(t−3)/(6−3)

The elasticity of the composition disclosed herein ranges from 10 to 80%, for example, from 15 to 70%, and further, for example, from 30 to 70%.

Based on its hardness and elasticity, the composition disclosed herein may exhibit good cohesion and can be easily used as make-up. The composition obtained may be very homogeneous and remain so even after application to the skin, for example, for several hours.

The presently disclosed composition has at least one excellent cosmetic property such as: it adheres sufficiently to the skin but not too strongly, it has a particularly soft feel, and/or it is easy to apply. Furthermore, such a composition can be very easily redispersible in water, which offers to the person using it the possibility of controlling the consistency and the covering power of their make-up according to the needs and/or the circumstances. Moreover, the composition makes it possible to obtain, after application to the skin and/or the mucous membranes, a very natural make-up finish. In addition, the particular texture of the composition makes it possible to apply it by the dry or wet route, such as with a sponge, and can give great suppleness in the softening during deposition with a wet sponge.

Finally, the composition is capable of being shaped by pressing, typically at a pressure of 0 (excluded) to about 50 bar, that is from 0 (excluded) to about 5×10⁶ Pa in a cosmetic container such as a cup or a dish or even a pot. Persons skilled in the art are capable of choosing the pressing pressure such that it allows shaping (the composition should take the shape of the cosmetic container) practically without expressing the liquid contained in the composition, i.e., practically without exudation.

According to another test which can characterize the novel texture of the composition according to the invention, such a composition is sufficiently solid to be left, in a cylindrical shape of approximately 3 cm³, on a horizontal plane at ambient atmospheric pressure (about 1.013 10⁵ Pa) and at room temperature (about 20° C.) without changing shape for one hour. “Solid” as used herein also means not flowable under its own weight for one hour.

Thus, such compositions can be advantageously shaped by pressing in a customary cosmetic container. The shaping by pressing generates a surface of contact between the product and the consumer (for example, the top surface of a dish, of a pot or even of a stick) enhancing the novel texture of the product, promoting contact with the product and demonstrating its pleasant (supple and soft) feel, giving an aesthetic appearance upon application: i.e., application with the finger, with a sponge, or with a brush (small or large).

Also disclosed herein is a make-up and/or care method comprising the at least partial application of the composition described above to any cutaneous zone of the body and/or of the face, such as to the skin, the eyelashes and the eyebrows, the mucous membranes (inside the lower eyelids) and the semimucous membranes (lips), and any other cutaneous zone of the body and of the face. Advantageously, the composition is therefore provided in the form of a foundation, blusher, eyebrow make-up, eyeshadow, concealer product, matt-effect care product or body make-up product composition intended to be at least partially applied to the face and/or to the body.

Other characteristics, aspects and advantages of the presently disclosed composition and methods will emerge on reading the detailed description which follows.

The expression “elastomeric” is understood to mean a deformable, supple material having viscoelastic properties and, for example, the consistency of a sponge or of a supple sphere. Its modulus of elasticity is such that this material withstands deformation and has a limited capacity to extension and to contraction. This material is capable of returning to its original shape after pulling. The elastomer is formed of polymeric chains of high molecular weight whose mobility is limited by a uniform network of crosslinking points.

The elastomeric solid organopolysiloxanes C of the composition disclosed herein generally do not dry the skin and can provide good cosmetic properties, such as softness and mattness. These novel elastomers may give compositions which can be pleasant upon application, which may spread well, and/or which can be soft and nonsticky to the touch. These compounds may confer good stability to water, upon application to the skin and/or the mucous membranes. Thus, the composition may have, in addition to at least one of the above advantages, good stability.

The solid elastomeric organopolysiloxanes C described herein are partially or completely crosslinked compounds and compounds with a three-dimensional structure. They are provided in the form of an aqueous dispersion of powder containing a solid elastomeric organopolysiloxane having a three-dimensional structure, dispersed in water. In the composition disclosed herein, the particles of organopolysiloxane C are contained in the at least one binder A, and not in the particulate phase B. As defined herein, the at least one binder A comprises all ingredients of the presently disclosed composition except for the particulate phase B. For example, the binder A may contain, in addition to the particles of organopolysiloxane C and water, at least one product capable of at least partially limiting the evaporation of water. This at least one product is discussed in detail below. In other embodiments, the binder A may contain only water and the particles of organopolysiloxane C, as well as minor amounts of impurities.

The dispersion (or suspension) of the particles is appreciably homogeneous.

The elastomeric organopolysiloxanes disclosed herein may be chosen from the crosslinked polymers described in application JP-A-10/175816. According to this document, the organopolysiloxanes are obtained by an addition and crosslinking reaction, in the presence of a catalyst, such as a platinum catalyst, of at least (i) one organopolysiloxane having at least two vinyl groups in the α- and ω-positions of the silicone chain per molecule, and (ii) one organopolysiloxane having at least one hydrogen atom bonded to a silicon atom per molecule.

The organopolysiloxane (i) may, for example, be chosen from polydimethylsiloxanes, such as an α,ω-dimethylvinylpoly-dimethylsiloxane.

The elastomeric organopolysiloxanes of the composition disclosed herein are advantageously provided in the form of an aqueous suspension. This suspension can be obtained, for example, as follows:

(a) mixing the organopolysiloxane (i) and the organopolysiloxane (ii);

(b) adding the aqueous phase comprising an emulsifier to the mixture of step (a);

(c) emulsifying the aqueous phase and the mixture;

(d) adding hot water to the emulsion of step (c); and

(e) polymerizing the organopolysiloxane (i) and the organopolysiloxane (ii) as an emulsion in the presence of a platinum catalyst.

The water is advantageously added at a temperature of 40-60 degrees C. After step (e), it is possible to dry the particles obtained, in order to evaporate therefrom all or part of the trapped water.

The elastomeric organopolysiloxane particles are in the form of deformable solid particles, which may optionally have a certain hardness, measurable with the Japanese JIS-A method. This JIS hardness can be measured on an elastomer block prepared for this purpose as follows: (1) mixing the organopolysiloxane (i) and the organosiloxane (ii);(2) removing the air from the mixture; (3) molding and vulcanizing the mixture in an oven at 100° C. for 30 minutes; (4) cooling the elastomer block to room temperature and (5) measuring the hardness. The relative density can also be determined on this elastomer block.

The JIS hardness may be less than or equal to 80, for example, less than or equal to 65. The organopolysiloxanes of the composition of the invention are, for example, those marketed under the names BY 29122, BY 29119, HMW 2220 and DC9509, by the company Dow-Corning Toray Silicone. It is also possible to use a mixture of these commercial products. An elastomer block based on the product BY-29122 has a hardness of 7 and based on the product BY-29119 a hardness of 30. The relative density is 0.97 to 0.98.

For example, the particles of the at least one elastomeric organopolysiloxane C (as active substance) have a size ranging from 0.1 to 500 μm, for example, from 0.1 to 200 μm. These particles may be spherical, flat or amorphous; for example, they may have a spherical shape.

The composition disclosed herein may contain, in addition, a fatty phase comprising at least one fatty substance chosen from fatty substances which are liquid at room temperature, called oils, such as those described in the document JP-A-10 175816, waxes and gums, which are generally solid at room temperature, and pasty fatty substances, wherein the oils, waxes, gums, and pasty fatty substances may be of animal, plant, inorganic or synthetic origin. In some embodiments of the presently disclosed composition, the fatty phase is part of the binder A. However, it is possible that when the fatty phase comprises a particulate fatty substance, this particulate fatty substance may be contained in the particulate phase B.

The oils useful herein may be chosen, for example, from silicone oils, fluorinated oils, fluorosilicone oils and hydrocarbon oils which are optionally partially siliconized. These oils may be volatile at room temperature and atmospheric pressure. The expression “volatile oil” is understood to mean, for instance, an oil capable of evaporating, in less than one hour, upon contact with the skin or the lips. The oil may be present in the composition in an amount ranging from 0.1 to 30%, such as from 0.1 to 15%, by weight relative to the total weight of the composition.

As oils which can be used in the composition herein, there may be mentioned in particular:

hydrocarbon oils of animal origin such as perhydrosqualene;

hydrocarbon vegetable oils such as liquid triglycerides of fatty acids, for example sunflower, maize, soybean, gourd, grapeseed, sesame, hazelnut, apricot, macadamia, castor, and avocado oils, triglycerides of caprylic/capric acids such as those sold by the company Stearineries Dubois or those sold under the names MIGLYOL 810, 812 and 818 by the company Dynamit Nobel;

the oils of formula R¹COOR² in which R¹ represents the residue of a higher fatty acid containing from 7 to 19 carbon atoms and R² represents a branched hydrocarbon chain containing from 3 to 20 carbon atoms such as for example Purcellin oil;

linear or branched hydrocarbons of inorganic or synthetic origin such as volatile or nonvolatile paraffin oils and derivatives thereof, petroleum jelly, polydecenes, hydrogenated polyisobutene such as parleam;

synthetic esters and ethers such as isopropyl myristate, octanoates, decanoates or ricinoleates of alcohols or of polyalcohols;

fatty alcohols such as octyl dodecanol or oleyl alcohol;

partially hydrocarbonaceous and/or siliconized fluorinated oils such as those described in the document JP-A-2-295912;

silicone oils such as polymethylsiloxanes containing a linear or cyclic silicone chain, which are liquid or pasty at room temperature, phenyl dimethicones, phenyl trimethicones and polymethylphenylsiloxanes; and

mixtures thereof.

In one embodiment, the composition disclosed herein comprises silicone oils. Such silicone oils may be amphiphilic, of the dimethicone copolyol type.

Advantageously, the composition may contain at least one wax chosen from hydrocarbon, fluorinated and silicone waxes, which may be solid or semisolid (in the form of a paste) at room temperature. The at least one wax may be of plant, mineral, animal and/or synthetic origin. In one embodiment, these waxes have a melting point greater than 25° C., for example, greater than 45° C.

The silicone waxes may be waxes containing a silicone structure and units containing one or more alkyl or alkoxy chains which are pendent and/or at the end of a silicone structure, these chains being linear or branched and containing from 10 to 45 carbon atoms. These waxes are called alkyldimethicones and alkoxydimethicones, respectively. Moreover, these alkyl chains may carry one or more ester functional groups.

As other waxes which can be used herein, there may be mentioned waxes chosen from at least one of: waxes of animal origin such as lanolin, beeswax; waxes of plant origin such as Carnauba or Candelilla wax; waxes of mineral origin, for example paraffin, lignite or microcrystalline waxes, ceresin or ozokerite; and synthetic waxes such as polyethylene waxes.

The waxes may be advantageously added in the form of an aqueous wax microdispersion, to provide, for example, staying power and/or mattness and/or to increase the cohesion of the pressed product in a cosmetic container such as a dish. Such a microdispersion of waxes is for example as described in patent EP-B 1-0,446,094. It may also be as marketed under the following trade references: POLYGEN PE and WE 1 by BASF, AQUACER 498 to 608 by BYK CERA, such as AQUACER 510 and AQUACER 608, or AQUACER 533 or alternatively SUPER OXYBRILL by Tiscco, or finally MICRODISPERSION 411 by Mocropowders.

These fatty substances may be chosen in a variety of ways by persons skilled in the art so as to prepare a composition having the desired properties, for example, of consistency or texture.

The composition may comprise, for example, from 0.1 to 30%, such as from 0.1 to 10% of wax, by weight relative to the total weight of the composition.

Advantageously, the composition disclosed herein may further comprise at least one aqueous phase gelling agent, namely a compound capable of giving the appearance of a gel to the composition and of thickening it. This gelling agent may be present in the composition in the quantities normally used, and for example in an amount of 0.1 to 20%, such as from 0.1 to 10%, by weight relative to the total weight of the composition.

The at least one aqueous phase gelling agent which may be used herein may be chosen from: water-soluble cellulosic gelling agents such as hydroxyethylcellulose, methylcellulose, hydroxypropylcellulose and carboxymethylcellulose; guar gum; quaternized guar gum; nonionic guar gums comprising C₁-C₆ hydroxyalkyl groups; xanthan, carob, scleroglucan, gellan and karaya gums; alginates, maltodextrin, starch and its derivatives, hyaluronic acid and its salts; clays, such as montmorillonites, hectorites or bentones, laponites; polymers containing a carboxyl group such as at least partially neutralized crosslinked polyacrylic acids such as “CARBOPOL” or “CARBOMER” from the company Goodrich (CARBOMER 980 for example neutralized with triethanolamine—abbreviated to TEA—); polyglyceryl (meth)acrylates; polyvinylpyrrolidone; polyvinyl alcohol; crosslinked polymers and copolymers of acrylamide; crosslinked homopolymers of methacryloyloxyethyltrimethylammonium chloride; associative polyurethanes, and associative polyamides.

For example, the at least one aqueous phase gelling agent may be chosen from xanthan gum, clays, associative polyurethanes, associative polyamides, cellulosic thickeners, in particular hydroxyethylcellulose, and at least partially neutralized crosslinked polyacrylic acids.

The composition disclosed herein may also comprise at least one product capable of at least partially limiting the evaporation of water, for example practically completely. Such a product can, for example, bind to water so as to at least partially prevent its evaporation. A composition into which such a product has been incorporated may have at least one of the following two properties, according to the measuring protocols detailed below: absence of production of drops inside a pot and/or a reduction in the water activity of the composition.

To measure these properties, a composition comprising 5% of this product is prepared. Two tests are applied, called respectively the drop test and the water activity test.

For the 1^{st} test, termed the “drop test,” 300 g of test composition are packaged immediately after manufacture in a glass pot having a capacity of 750 ml, an outer diameter of 98 mm±1 mm and a height of 142 mm±1 mm of the glass pot type having the trade reference “Bocal Twist Off 750 ml, H02768” from Société Parisienne de Verrerie. This pot is closed with a metal lid having an inner diameter of 82 mm which screws onto the top of the “82 mm white metal cover, R27182”, from Société Parisienne de Verrerie, type. It is ensured that the jar is properly closed with the lid by tightening it sufficiently, and preferably it is ensured that the jar is leaktight by surrounding the cover plus at the minimum 3 cm from the top of the jar with a stretchable laboratory plastic film of the Parafilm® type from the company American National Can. The combination “formula in jar closed with a cover+Parafilm®” is placed at room temperature (20-25° C.), at atmospheric pressure (1.013×10⁵ Pa). The inner walls of the jar are observed every 24 h between t=0 and t=10 days. If no water droplets can be perceived with the naked eye on the walls during this period of observation, the product is a product which can be used in the composition herein.

For the second test, termed The “water activity test,” the water activity is measured on a ROTRONIC type apparatus. This ROTRONIC apparatus consists of three main members: a water bath, a measuring sensor, and a system for reading the measurement. The values given were obtained using the members of the following types: thermostated water bath Ecoline RE 204 LAUDA, Hygrometre AwV C ROTRONIC (measuring sensor) connected to the thermostated water bath, and a Hygroscope BT-RS1 ROTRONIC (reading of the measurement of water activity) connected to the hygrometer. The sample (about 3 g) is placed in a PS14 type single use sample cuvette (diameter 46 mm×16 mm). The sample and cuvette combination is placed in a measuring chamber which is thermostatted at 23° C. The combination is thus allowed to stabilize in temperature for 30 minutes after which the water activity or Aw value is read. If the Aw value is less than or equal to 0.95±0.005, the product is a product which can be used in the composition herein.

Such a product is for example chosen from sugars such as trehalose, and polyols and glycols such as glycerin, polyethylene glycol and propylene glycol. For example, the product may be glycerin. Such a product is present in the composition disclosed herein in an amount ranging, for example, from 0.1 to 30%, such as from 0.1 to 10%, by weight relative to the total weight of the composition.

The composition disclosed herein may be provided in the form of a paste or a powder, which is, for example, compact, loose or pressed.

The composition may be provided in the form of a foundation, blusher, eyeshadow, eyebrow make-up, concealer product or mattifying product composition intended to be at least partially applied to the face or to the body.

Of course, the composition should be cosmetically or dermatologically acceptable, namely nontoxic and capable of being applied to the skin (including the inside of the eyelids) or the lips of human beings.

The composition disclosed herein also comprises least one particulate phase B which may comprise at least one pigment and/or at least one pearlescent agent and/or at least one filler and/or glitter, which are normally used in cosmetic compositions, and/or mixtures thereof, which serve to color and/or opacify and/or give body to the composition so as to promote its make-up and/or care use. The expression “particulate phase” is understood to mean a phase comprising components in the form of particles, which may be of different sizes and/or of different types, and, for example, practically essentially consisting of such particles. Further, as defined herein, the particulate phase does not include the particles of the polyorganosiloxane C. These particles may be spherical, flat, in the form of needles or with no differentiated shape.

The particles of the particulate phase may be coated with at least one silicone compound such as polydimethylsiloxanes and/or with polymers, such as polyethylenes or polymethacrylates, and/or at least one fluorinated compound and/or at least one amino acid and/or one mineral coating in the form of a continuous or discontinuous layer or of particles such as, for example, silica.

The expression “pigment” should be understood to mean at least one white or colored, inorganic or organic particle, insoluble in the medium and intended to color and/or opacify the composition. The pigment may be present in an amount ranging, for example, from 0.05 to 60% by weight, such as from 0.5 to 50%, relative to the total weight of the composition. It may be of a customary or nanometric size. Inorganic pigments may be chosen from at least one of titanium, zirconium and cerium dioxides, zinc, iron and chromium oxides, nanotitaniums, nanozincs, and ferric blue. The organic pigments may be chosen from carbon black, lacquers such as calcium, barium, aluminium and zirconium salts, and acid dyes such as haloacid, azo and anthraquinone dyes. It is also possible to envisage mixing at least one inorganic pigment and at least one organic pigment.

The pigment may, for example, be coated with at least one silicone compound such as a polydimethylsiloxane and/or with polymers, such as polyethylene, or alternatively with an inorganic compound, for example in the form of a continuous or discontinuous layer or of particles such as silica. It is thus possible to mention the “SI oxides” which are pigments coated with polymethylhydrogenosiloxanes which are sold by the company Myioshi.

The expression “fillers” should be understood to mean colorless or white, mineral or synthetic particles intended to give body or stiffness to the composition, and/or softness, and/or mattness and/or uniformity to the make-up, and to contribute to good disintegration of the composition during taking and application.

At least one filler may be present in the particulate phase in an amount ranging, for example, from 0.05 to 60% by weight, such as from 20% to 60%, by weight relative to the total weight of the composition. It may be inorganic or synthetic. The at least one filler may be chosen from at least one of talc, for example, talc which is surface-treated to make it hydrophilic, mica, silica, kaolin, Nylon® powder (such as Orgasol® from Atochem), polyethylene powder, Teflon®, sericites, clays, starch, boron nitride, powders of tetrafluoroethylene polymers, powders of polymethyl methacrylate, polyurethane powders such as BPD-500 from the company KOBO, polystyrene powders, polyester powders, synthetic hollow microspheres such as Expancel® (from the company NOBEL INDUSTRIE), microsponges such as Polytrap® (from the company DOW CORNING) and microbeads of polymethylsilsesquioxane resin (Tospearl® from the company TOSHIBA, for example), zinc and titanium oxides, zirconium and cerium oxides, precipitated calcium carbonate, magnesium carbonate and hydrocarbonate, hydroxyapatite, hollow silica microspheres (Silica Beads® from the company MAPRECOS), glass and ceramic microcapsules, and metal soaps derived from organic carboxylic acids having from 8 to 22 carbon atoms, such as from 12 to 18 carbon atoms, per molecule, such as zinc, magnesium and lithium stearate, zinc laurate, magnesium myristate. For example, the at least one filler may be chosen from mica, talc (such as hydrophilic talc), synthetic hollow microspheres, polyurethane powder and Nylon® powders. The at least one filler may also be chosen from polyamide fibers, such as Nylon® fibers.

The expression “pearlescent agents” should be understood to mean the iridescent particles which reflect light. At least one pearlescent agent may be present in the composition in an amount ranging, for example, from 0.05 to 60% by weight, such as from 10 to 50% by weight, relative to the total weight of the composition. The at least one pearlescent agent may be chosen from: natural pearl; mica coated with titanium oxide, with iron oxide, with aluminium hydroxide, with magnesium hydroxide, with silica, with a natural pigment and with bismuth oxychloride; and colored mica-titanium.

The compositions disclosed herein may also comprise glitter.

In one embodiment, the particulate phase comprises at least one compound chosen from the group consisting of mica, pearlescent agents, preferably hydrophilic talc, synthetic hollow microspheres, polyurethane powder, pigments and Nylon® powders.

The composition disclosed herein may also comprise at least one water-soluble colorant, such as beet juice and/or methylene blue, which may be present in an amount ranging from 0.05 to 6% by weight relative to the total weight of the composition.

The composition may also comprise at least one preservative which is commonly used by persons skilled in the art, such as phenoxyethanol, parabens, chlorophenesin, benzyl alcohol, chlorhexidine gluconate, mixture of DMDM/HMDM hydantoin+butan-1,3-diol+iodopropynylbutylcarbamate (70/4.5/2.5) in water (of the Glidant plus liquid type from LONZA), an ethyl alcohol+pentylene glycol+sodium methylparaben mixture preferably in the proportions 47/47/6 as described in European patent application EP-0,935,960, a pentylene glycol+sodium methylparaben mixture, and mixtures thereof, in an amount ranging from 0 (excluded) to a percentage which is sufficient to protect the formula, as is known to persons skilled in the art, generally at the maximum 10%, by weight relative to the total weight of the composition, for example in an amount ranging from 0 (excluded) to 2%, by weight relative to the total weight of the composition.

According to one embodiment, the composition comprises, by weight relative to the total weight of the composition:

from 25 to 45% of at least one organopolysiloxane C and

from 25 to 60% of at least one particulate phase B

from 15 to 35% of an aqueous phase D.

wherein the weight ratio of (said at least one organopolysiloxane C+said aqueous phase D) to said at least one particulate phase B ranges from 0.6:1 to 3:1. In one embodiment, the weight ratio of (C+D)/B ranges from 0.6:1 to 2.5:1, for example, from 1:1 to 2.5:1. The 15 to 35% of the aqueous phase D does not contain any particles of organopolysiloxane C, but may, in some embodiments, contain other ingredients in addition to water. In some embodiments, the organopolysiloxane C+the aqueous phase D together may be equal to the binder A discussed above. The weight ratio C/B of the at least one organopolysiloxane C to the at least one particulate phase B ranges from 0.4:1 to 1.8:1, such as from 0.4:1 to 1:5:1, for example, from 0.4:1 to 1.3:1, and further, for example, from 0.6:1 to 1.3:1.

In another embodiment, the composition may also be described as comprising by weight, relative to the total weight of the composition:

from 40 to 75% of at least one binder A, wherein the binder A comprises 63% organopolysiloxane C and 37% water, and

from 25 to 60% of at least one particulate phase B,

such that the weight ratio of A/B ranges from 0.6:1 to 3:1, for example, from 0.6:1 to 2.5:1, and further for example, from 1:1 to 2.5:1.

Of course, persons skilled in the art will be careful to choose the possible additional additives and/or their quantity such that the advantageous properties of the presently disclosed composition are not, or not substantially, impaired by the addition envisaged. These additives, for example, should not interfere with the homogeneity, stability, comfort and freshness of the composition.

The composition may be manufactured in the cold state or by heating at least one elastomeric organopolysiloxane in powdered form dispersed in water, adding a particulate phase, for example one or more pigments and/or one or more fillers, and/or at least one other compound, optionally adding a fatty phase in the liquid state (in particular heated to the highest melting point of the waxes), followed by homogenization, for example, emulsification, if necessary.

It may also be obtained by blending/extrusion as described in patent application EP-A-667 146. This method comprises blending the paste (binders+additives+fillers+pigments) during the cooling with the aid of a roll mill or a twin-screw extruder-mixer, such as the cooker-extruder mixer “BC21” from the company “CLEXTRAL”. This method allows the production of a composition in the form of a deformable consistent paste.

A composition as disclosed herein with contents of particulate phase greater than 85% may also be prepared using the mixer-granulators which are customary for persons skilled in the art, for example of the Baker or Lödige trademark.

One method of preparing the composition disclosed herein comprises the use of at least one method of blending, preferably a twin-screw extrusion blending. One embodiment of this method of preparation, with the use of a twin-screw extruder, such as the cooker-extruder mixer “BC21” from the company “CLEXTRAL”, comprises introducing a particulate phase in pulverulent form at the top of a twin-screw extruder by at least one means for introducing the said phase, for example a metering device of the weight metering device type, introducing an aqueous suspension of organopolysiloxane by at least one means of introducing the suspension, for example a pump of the peristaltic pump type, followed by cold blending in the extruder. The supple roll thus obtained at the outlet of the extruder is then collected, shaped, for example, in a dish and then pressed, at a pressure sufficient to allow this shaping without expressing the liquid contained in the material forming the roll, so as to take the shape of the said dish.

Regardless of their physical state (liquid or pulverulent), the additives (all or some), such as the preservative, may be introduced into the extruder in three different ways. A first way comprises using a feed independent of that of the particulate phase and of that of the aqueous suspension: the additives in liquid form are introduced by a peristaltic pump into the appropriate barrels; the pulverulent additives are introduced by a powder weight metering device into the appropriate barrels. A second way comprises feeding the said additive into the aqueous suspension provided an excessively strong interaction therewith is not brought about, as is known to persons skilled in the art. For example, such an interaction may be a collecting into a mass or an increase in viscosity which rules out pumping of the mixture. Finally, according to a third way, in the case of an interaction with the crosslinked aqueous suspension and provided the amount of additive is less than 15%, it can be advantageously incorporated into the pulverulent phase. The mixing of the pulverulent phase and of the additive, such as a preservative, may be carried out in a Baker type powder mixer and generally takes place before the extrusion/blending step. The mixture obtained at the outlet of the Baker device has the appearance of a loose powder and may be introduced into the extruder/blender via a powder weight metering device of the K-TRON metering device type.

The composition disclosed herein, in this case, is in the form of a consistent paste with a powdered appearance.

The examples of compositions below are given by way of illustration of the invention without any limitation.

### EXAMPLES OF IMPLEMENTATION

The compositions described below were prepared, with the exception of the third make-up formula and the second care formula, according to the following procedure for manufacture on a corotating twin-screw extruder, which is a cooker-extruder mixer “BC21” from the company “CLEXTRAL”:

introducing the pearlescent agents and/or pigments and/or fillers in pulverulent form at the head of the extruder by a weight metering device,

introducing the aqueous suspension of crosslinked polydimethylsiloxane (BY-29119 from the company Dow Corning) by a peristaltic pump into one of the first three barrels of the extruder,

introducing the other optional additives in liquid form into the appropriate barrels, for example by peristaltic pumps, and

blending in the cold state in the extruder.

The supple roll obtained at the outlet of the extruder was manually collected, placed in a dish and pressed so as to take the shape of the dish. The pressure used allowed this shaping without expressing the liquid contained in the roll material.

### EXAMPLE 1

A first pearlescent formula according to the composition disclosed herein had the following composition:

| | |
|---|---|
| Particulate Phase B: | |
| sodium salt of Ponceau | 0.06% |
| mica-titanium oxide/brown iron oxide | 40% |
| quinoline yellow | 0.06% |
| Binder A: | 59.88% |
| BY-29119* | |

*BY-29119 contains 63% of active substance, i.e., 63% of hydrophilic organopolysiloxane powder and 37% water. For purposes of this example only, therefore, BY-29119 is defined as binder A..

The BY-29119/particulate phase weight ratio (i.e., A/B) is 1.49:1, and the organosiloxane/particulate phase (i.e., C/B) weight ratio is 0.94:1. The organopolysiloxane C, contained in BY-29119 together with water, comprised 63% of the 59.88% of the total composition comprised by the BY-29119. The particulate phase B comprised mica, titanium oxide, and brown iron oxide, in addition to the sodium salt of Ponceau and quinoline yellow.

A coppery-brown colored eyeshadow, which was very pleasant to the touch and which gave a sensation of freshness once applied to the skin of the eyelids, was thus obtained. The hardness of this composition was 0.07 N (standard deviation±0.006) and the elasticity was 17.778% (standard deviation±1.26).

### EXAMPLE 2

A second formula according to the composition disclosed herein had the following composition:

| | |
|---|---|
| Particulate Phase B: | |
| titanium oxide (untreated anatase) | 6.74% |
| yellow iron oxide | 1.57% |
| brown, yellow iron oxide | 1.35% |
| black iron oxide | 0.34% |
| nylon powder (Orgasol ® from Atochem) | 30% |
| Binder A: | 60% |
| BY-29119* | |

*BY-29119 contains 63% of active substance, i.e., 63% of hydrophilic organopolysiloxane powder and 37% water. For purposes of this example only, therefore, BY-29119 is defined as binder A..

The BY-29119/particulate phase weight ratio (i.e., A/B) is 1.5:1, and the organopolysiloxane (as 63% of the BY-29119)/particulate phase (i.e., C/B) weight ratio is 0.945:1. The particulate phase B comprised titanium oxide (untreated anatase), yellow iron oxide, brown, yellow iron oxide, black iron oxide, and nylon powder (Orgasol® from Atochem) as set forth above.

A foundation, which was very pleasant to the touch and which gave a sensation of freshness once applied to the skin of the face, was thus obtained. The make-up thus prepared was particularly remarkable by its comfortable, soft, natural and powdered features. The hardness of this composition was 0.123 N (standard deviation±0.003) and the elasticity was 40.333% (standard deviation±1.2).

### EXAMPLE 3

A third formula according to the composition disclosed herein had the following composition:

| | |
|---|---|
| Particulate Phase B: | |
| titanium oxide (untreated anatase) | 6.74% |
| yellow iron oxide | 1.57% |
| yellow, brown iron oxide | 1.35% |
| black iron oxide | 0.34% |
| Nylon powder (Orgasol ® from Atochem) | 25% |
| Spherical and porous mica coated with | 5% |
| silica (50/30/20)(Velvetveil H6400 | |
| from the company CATALYSTS & CHEMICALS) | |
| Binder A: | |
| BY-29119* | 54% |
| glycerine | 5% |
| preservative Phenochem from the | 1% |
| company Sharon Lab. | |

*BY-29119 contains 63% of active substance, i.e., 63% of hydrophilic organopolysiloxane powder and 37% water. However, for purposes of this example, BY-29119 is NOT defined as binder A, since the glycerine and preservative.are also contained in binder A.

The BY-29119/particulate phase weight ratio is 1.35:1, and the organopolysiloxane (as 63% of the BY-29119)/particulate phase weight ratio (i.e., C/B) is 0.85:1. The particulate phase comprises titanium oxide (untreated anatase), yellow iron oxide, brown, yellow iron oxide, black iron oxide, nylon powder (Orgasol® from Atochem), and spherical and porous mica coated with silica (50/30/20)(Velvetveil H6400). The binder A is present at 60% (54% BY-29119+5% glycerine+1% preservative) so the A/B ratio is 1.5:1.

This composition was prepared in two different ways: either by mixing glycerine-preservative and feeding the extruder-blender via a peristaltic pump, or by introducing the preservative into the pulverulent phase as is detailed in the description as a third way of introducing the additive, here the preservative.

A foundation, which was very pleasant to the touch and which gave a sensation of freshness once applied to the skin of the face, was thus obtained regardless of the mode of implementation among the two modes of implementation described above. The hardness of this composition was 0.164 N (standard deviation±0.003) and the elasticity was 30.222% (standard deviation±1.02). The texture of this composition differed from the composition of the preceding example in its “creamy” feel and its appearance of great homogeneity, the make-up thus prepared was also remarkable on the same criteria.

The three make-up compositions described above thus had remarkable properties of suppleness on taking, of pleasantness on application and of freshness during application. Furthermore, such compositions made it possible to easily control the covering power and the intensity of the deposit, by virtue of their consistency which can be adjusted by dilution during use, which makes them “tailor-made” products which are highly valued by users.

### EXAMPLE 4

Likewise, it is possible to manufacture mattifying care compositions according to the composition disclosed herein. Such care compositions, for example, have the compositions of the preceding Examples 2 and 3, in which the pigments and colorant have been replaced with Nylon powder.

The following mattifying care compositions were thus obtained:

First mattifying care formula:

| | |
|---|---|
| Nylon powder (Orgasol ® from Atochem) | 40% |
| BY-29119* | 60% |

*BY-29119 contains 63% of active substance, i.e., 63% of hydrophilic organopolysiloxane powder and 37% water. For purposes of this example only, therefore, BY-29119 is defined as binder A..

A mattifying care product was thus obtained which was very pleasant to the touch and which gave a sensation of freshness once applied to the skin of the face.

The BY-29119/particulate phase (since the only ingredient in the particulate phase is the nylon powder (Orgasol® from Atochem), the nylon powder=the particulate phase in this example) weight ratio (A/B) is 1.5:1, and the organopolysiloxane (as 63% of the BY-29119)/particulate phase (C/B) weight ratio is 0.945:1. The hardness of this composition was 0.264 N (standard deviation±0.003) and the elasticity was 41.333% (standard deviation±1.0).

Second mattifying care formula:

| | |
|---|---|
| Particulate Phas B: | |
| Nylon powder (Orgasol ® from Atochem) | 35% |
| Velvetveil H6400 | 5% |
| Binder A: | |
| BY-29119* | 54% |
| glycerine | 5% |
| preservative Phenochem from the | 1% |
| company Sharon Lab. | |

*BY-29119 contains 63% of active substance, i.e., 63% of hydrophilic organopolysiloxane powder and 37% water. However, for purposes of this example, BY-29119 is NOT defined as binder A, since the glycerine and preservative.are also contained in binder A.

The BY-29119 binder/particulate phase (nylon powder (Orgasol® from Atochem) plus Velvetveil H6400) weight ratio (A/B) is 1.35:1, and the organopolysiloxane (as 63% of the BY-29119)/particulate phase (C/B) weight ratio is 0.85:1. The binder A is present at 60% (54% BY-29119+5% glycerine+1% preservative) so the A/B ratio is 1.5:1.

A mattifying care product was thus obtained which was very pleasant to the touch and which gave a sensation of freshness once applied to the skin of the face. This second composition differed from the preceding one by its more creamy appearance and a greater homogeneity of its appearance in a dish. The hardness of this composition was 0.325 N (standard deviation±0.011) and the elasticity was 35.333% (standard deviation±0.33).

### EXAMPLE 5

A formulation of make-up for the face was prepared according to the composition disclosed herein and having the following composition:

| Particulate Phase B: | |
|---|---|
| titanium oxide (untreated anatase) | 8.1% |
| yellow iron oxide | 1.2% |
| yellow, brown iron oxide | 0.55% |
| black iron oxide | 0.15% |
| Nylon powder (Orgasol ® from Atochem) | 10% |
| Polyamide Fibers (0.9 DTEX, length 0.3 mm) | 5% |
| from Paul Bonte (INCI name = Nylon-66) | |
| POLYTRAP 603 ULTRA-LITE SORPTION | 5% |
| POLYMER POWDER from A.P.S. (INCI name = | |
| Acrylates Copolymer) | |
| Binder A: | |
| BY-29119* | 59% |
| glycerine | 5% |
| preservative Phenochem from the | 1% |
| company Sharon Lab. | |
| propylene glycol | 5% |

*BY-29119 contains 63% of active substance, i.e., 63% of hydrophilic organopolysiloxane powder and 37% water. However, for purposes of this example, BY-29119 is NOT defined as binder A, since the glycerin, propylene glycol, and preservative.are also contained in binder A.

The BY-29119/particulate phase weight ratio is 1.35:1, and the organopolysiloxane (as 63% of the BY-29119)/particulate phase weight ratio (i.e., C/B) is 1.2:1. The particulate phase comprises titanium oxide (untreated anatase), yellow iron oxide, brown, yellow iron oxide, black iron oxide, nylon powder (Orgasol® from Atochem), nylon fibers (nylon-66) and the POLYTRAP microsponges. The binder A is present at 70% (59% BY-29119+5% glycerine+1% preservative+5% propylene glycol) so the A/B ratio is 2.3:1.

### EXAMPLE 6

An eyeshadow formulation was prepared according to the composition disclosed herein and having the following composition:

| | |
|---|---|
| Particulate Phase B: | 49% |
| Pearls (mica/titanium dioxide ± iron oxide ± | |
| ferric ferrocyanide ± carmine) | |
| Binder A: | |
| BY-29119* | 45% |
| glycerine | 5% |
| preservative Phenochem from the | 1% |
| company Sharon Lab. | |

*BY-29119 contains 63% of active substance, i.e., 63% of hydrophilic organopolysiloxane powder and 37% water. However, for purposes of this example, BY-29119 is NOT defined as binder A, since the glycerin and preservative.are also contained in binder A.

The BY-29119/particulate phase weight ratio is 0.92:1, and the organopolysiloxane (as 63% of the BY-29119)/particulate phase weight ratio (i.e., C/B) is 0.6:1. The particulate phase comprises the pearls listed above. The binder A is present at 51% (45% BY-29119+5% glycerine+1% preservative) so the A/B ratio is 1:1.

What is claimed is:

1. A cosmetic composition comprising at least one binder A and at least one particulate phase B, the binder A comprising at least water and particles of at least one partially or completely crosslinked elastomeric solid organopolysiloxane C, wherein the weight ratio of said at least one organopolysiloxane C to said at least one particulate phase B ranges from 0.4:1 to 1.8:1.

2. A composition according to claim 1, wherein the weight ratio of said at least one organopolysiloxane C to said at least one particulate phase B ranges from 0.4:1 to 1.5:1

3. A composition according to claim 2, wherein the weight ratio of said at least one organopolysiloxane C to said at least one particulate phase B ranges from 0.4:1 to 1.3:1.

4. A composition according to claim 3, wherein the weight ratio of said at least one organopolysiloxane C to said at least one particulate phase B ranges from 0.6:1 to 1.3:1.

5. A composition according to claim 1, wherein the weight ratio of said at least one binder A to said at least one particulate phase B ranges from 0.6:1 to 3:1.

6. A composition according to claim 5, wherein the weight ratio of said at least one binder A to said at least one particulate phase B ranges from 0.6:1 to 2.5:1.

7. A composition according to claim 6, wherein the weight ratio of said at least one binder A to said at least one particulate phase B ranges from 1:1 to 2.5:1.

8. A composition according to claim 1, wherein said at least one particulate phase B is present in said composition in an amount ranging from 25 to 60% by weight, relative to the total weight of the composition.

9. A composition according to claim 8, wherein said at least one particulate phase B is present in said composition in an amount ranging from 35 to 60% by weight, relative to the total weight of the composition.

10. A composition according to claim 9, wherein said at least one particulate phase B is present in said composition in an amount ranging from 35 to 55% by weight, relative to the total weight of the composition.

11. A composition according to claim 1, wherein said at least one organopolysiloxane C is present in said composition in an amount ranging from 25 to 45% by weight, relative to the total weight of the composition.

12. A composition according to claim 1, wherein the particles of said at least one organopolysiloxane C have a particle size ranging from 0.1 to 500 μm.

13. A composition according to claim 12, wherein the particles of said at least one organopolysiloxane C have a particle size ranging from 0.1 to 200 μm.

14. A composition according to claim 1, wherein the particles of said at least one organopolysiloxane have a JIS hardness of less than or equal to 80.

15. A composition according to claim 1, wherein the particles of said at least one organopolysiloxane have a JIS hardness of less than or equal to 65.

16 A composition according to claim 1, wherein said at least one particulate phase B comprises at least one particle chosen from pigments, pearlescent agents, fillers, and glitter.

17. A composition according to claim 16, wherein said at least one particulate phase B comprises at least one pigment.

18. A composition according to claim 17, wherein said at least one pigment is chosen from titanium dioxides, zirconium dioxides, cerium dioxides, zinc oxides, iron oxides, chromium oxides, nanotitaniums, nanozincs, ferric blue, carbon black, and lacquers.

19. A composition according to claim 17, wherein said at least one pigment is present in an amount ranging from 0.05% to 60% by weight, relative to the total weight of the composition.

20. A composition according to claim 19, wherein said at least one pigment is present in an amount ranging from 0.5 to 50% by weight, relative to the total weight of the composition.

21. A composition according to claim 16, wherein said at least one particulate phase B comprises at least one pearlescent agent.

22. A composition according to claim 21, wherein said at least one pearlescent agent is chosen from natural pearl, mica coated with titanium oxide, mica coated with iron oxide, mica coated with aluminium hydroxide, mica coated with magnesium hydroxide, mica coated with silica, mica coated with a natural pigment and mica coated with bismuth oxychloride, and colored mica-titanium.

23. A composition according to claim 21, wherein said at least one pearlescent agent is present in an amount ranging from 0.05% to 60% by weight, relative to the total weight of the composition.

24. A composition according to claim 22, wherein said at least one pearlescent agent is present in an amount ranging from 10% to 50% by weight, relative to the total weight of the composition.

25. A composition according to claim 16, wherein said at least one particulate phase B comprises at least one filler.

26. A composition according to claim 25, wherein said at least one filler is chosen from talc, mica, silica, kaolin, Nylon® powders, polyethylene powder, sericites, clays, starch, boron nitride, powders of tetrafluoroethylene polymers, powders of polymethyl methacrylate, polyurethane powders, polystyrene powders, polyester powders, synthetic hollow microspheres, microsponges and microbeads of polymethylsilsesquioxane resin, zinc and titanium oxides, zirconium and cerium oxides, precipitated calcium carbonate, magnesium carbonate and hydrocarbonate, hydroxyapatite, hollow silica microspheres, glass and ceramic microcapsules, and metal soaps of organic acids having from 8 to 22 carbon atoms.

27. A composition according to claim 26, wherein said at least one filler is chosen from mica, talc, Nylon® powders, synthetic hollow microspheres and polyurethane powders.

28. A composition according to claim 25, wherein said at least one filler is present in an amount ranging from 0.05% to 60% by weight, relative to the total weight of the composition.

29. A composition according to claim 28, wherein said at least one filler is present in an amount ranging from 20% to 60% by weight, relative to the total weight of the composition.

30. A composition according to claim 1, wherein said at least one particulate phase B comprises particles chosen from at least one of pearlescent agents, pigments, mica, talc, synthetic hollow microspheres, polyurethane powder and Nylon® powders.

31. A composition according to claim 16, wherein said at least one particle is coated with at least one compound chosen from silicone compounds, a polyethylene, a polymethacrylate, a fluorinated compound, an amino acid, and silica.

32. A composition according to claim 1, wherein said composition further comprises at least one fatty substance chosen from oils, waxes, gums, and pasty fatty substances, wherein each at least one fatty substance may be of animal, plant, mineral or synthetic origin.

33. A composition according to claim 32, wherein said composition comprises an aqueous microdispersion of wax.

34. A composition according to claim 32, wherein said composition comprises at least one wax in an amount ranging from 0.1% to 30% by weight, relative to the total weight of the composition.

35. A composition according to claim 34, wherein said composition comprises at least one wax in an amount ranging from 0.1% to 10% by weight, relative to the total weight of the composition.

36. A composition according to claim 32, wherein said composition comprises at least one oil in an amount ranging from 0.1% to 30% by weight, relative to the total weight of the composition.

37. A composition according to claim 36, wherein said composition comprises at least one oil in an amount ranging from 0.1% to 15% by weight, relative to the total weight of the composition.

38. A composition according to claim 32, wherein said composition comprises a volatile oil.

39. A composition according to claim 1, wherein said composition further comprises at least one aqueous phase gelling agent.

40. A composition according to claim 39, wherein said at least one aqueous phase gelling agent is chosen from water-soluble cellulosic gelling agents; guar gum; quaternized guar gum; nonionic guar gums comprising C₁-C₆ hydroxyalkyl groups; xanthan, carob, scleroglucan, gellan and karaya gums; alginates, maltodextrin, starch and its derivatives, hyaluronic acid and its salts; clays; at least partially neutralized crosslinked polyacrylic acids; polyglyceryl (meth)acrylate polymers; polyvinylpyrrolidone; polyvinyl alcohol; crosslinked polymers and copolymers of acrylamide; crosslinked homopolymers of methacryloyloxyethyltrimethylammonium chloride; associative polyurethanes, and associative polyamides.

41. A composition according to claim 39, wherein said at least one aqueous phase gelling agent is chosen from xanthan gum, clays, associative polyurethanes, associative polyamides, cellulosic thickeners, and at least partially neutralized crosslinked polyacrylic acids.

42. A composition according to claim 39, wherein the at least one aqueous phase gelling agent is present in an amount ranging from 0.1% to 20% by weight, relative to the total weight of the composition.

43. A composition according to claim 42, wherein the at least one aqueous phase gelling agent is present in an amount ranging from 0.1% to 10% by weight, relative to the total weight of the composition.

44. A composition according to claim 1, wherein said composition further comprises at least one product capable of at least partly limiting the evaporation of water.

45. A composition according to claim 44, wherein said at least one product is chosen from sugars and polyols.

46. A composition according to claim 45, wherein said at least one product is chosen from glycerine, trehalose, polyethylene glycol and propylene glycol.

47. A composition according to claim 44, wherein said at least one product is present in an amount of 0.1% to 30% by weight, relative to the weight of the composition.

48. A composition according to claim 47, wherein said at least one product is present in an amount of 0.1% to 10% by weight, relative to the weight of the composition.

49. A composition according to claim 1, wherein said composition comprises by weight, relative to the total weight of the composition:
from 25 to 60% of said at least one particulate phase B,
from 25 to 45% of said at least one organopolysiloxane C, from 15 to 35% of an aqueous phase D,
wherein the weight ratio of (said at least one organopolysiloxane C+said aqueous phase D) to said at least one particulate phase B ranges from 0.6:1 to 3:1.

50. A composition according to claim 49, wherein the weight ratio of (said at least one organopolysiloxane C+said aqueous phase D) to said at least one particulate phase B ranges from 0.6:1 to 2.5:1.

51. A composition according to claim 1, wherein said composition comprises by weight, relative to the total weight of the composition:
from 40 to 75% of said at least one binder A, wherein the binder A comprises 63% organopolysiloxane C and 37% water, and
from 25 to 60% of said at least one particulate phase B,
wherein the weight ratio of said at least one binder A to said at least one particulate phase B ranges from 0.6:1 to 3:1.

52. A composition according to claim 51, wherein the weight ratio of said at least one binder A to said at least one particulate phase B ranges from 0.6:1 to 2.5:1.

53. A composition according to claim 52, wherein the weight ratio of said at least one binder A to said at least one particulate phase B ranges from 1:1 to 2.5:1.

54. A cosmetic composition comprising at least one binder A and at least one particulate phase B, the binder A comprising at least water and particles of at least one partially or completely crosslinked elastomeric solid organopolysiloxane C, wherein the weight ratio of said at least one binder A to said at least one particulate phase B ranges from 0.6:1 to 3:1.

55. A composition according to claim 54, wherein the weight ratio of said at least one binder A to said at least one particulate phase B ranges from 0.6:1 to 2.5:1.

56. A composition according to claim 55, wherein the weight ratio of said at least one binder A to said at least one particulate phase B ranges from 1:1 to 2.5:1.

57. A composition according to claim 1, wherein the composition is sufficiently solid to be left, in a cylindrical form of 3 cm³, on a horizontal plane at ambient atmospheric pressure and at room temperature without changing shape for one hour.

58. A composition according to claim 1, wherein said composition is provided in the form of a foundation, blusher, eyebrow make-up, eyeshadow, concealer product, body make-up product or matt-effect care product composition.

59. A method for manufacturing a cosmetic composition, wherein the cosmetic composition comprises at least one binder A and at least one particulate phase B, the binder A comprising at least water and particles of at least one partially or completely crosslinked elastomeric solid organopolysiloxane C, wherein the weight ratio of said at least one organopolysiloxane C to said at least one particulate phase B ranges from 0.4:1 to 1.8:1, and wherein the composition is obtained by blending and/or extrusion.

60. A method according to claim 59, wherein the blending is carried out with the aid of a twin-screw extruder-mixer.

61. A method according to claim 59, wherein the composition is shaped by pressing in a container.

62. A cosmetic method for applying make-up to or caring for the skin, the lips, the eyelashes, the eyebrows, said method comprising applying to the skin, the lips, the eyelashes or the eyebrows a composition comprising at least one binder A and at least one particulate phase B, the binder A comprising at least water and particles of at least one partially or completely crosslinked elastomeric solid organopolysiloxane C, wherein the weight ratio of said at least one organopolysiloxane C to said at least one particulate phase B ranges from 0.4:1 to 1.8:1.

63. A cosmetic method for applying make-up to the skin with a sensation of freshness, said method comprising applying to the skin a composition comprising at least one binder A and at least one particulate phase B, the binder A comprising at least water and particles of at least one partially or completely crosslinked elastomeric solid organopolysiloxane C, wherein the weight ratio of said at least one organopolysiloxane C to said at least one particulate phase B ranges from 0.4:1 to 1.8:1.
